(19) United States
(12) Patent Application Publication
(10) Pub. No.: US 2008/0271735 A1
(43) Pub. Date: Nov. 6, 2008

(54) Artificial airway

(76) Inventor: Sherry Luken, Georgetown, TX (US)

Correspondence Address: TROP PRUNER & HU, PC, 1616 S. VOSS ROAD, SUITE 750, HOUSTON, TX, 77057-2631, US

(21) Appl. No.: 12/151,190

(22) Filed: May 5, 2008

Related U.S. Application Data
(60) Provisional application No. 60/927,629, filed on May 4, 2007.

(51) Int. Cl. A61M 16/00; A61J 17/00

(52) U.S. Cl. 128/200.26; 606/234

(57) ABSTRACT

One embodiment of the invention includes a pediatric pacifier with a passage. An oropharyngeal airway may be inserted through the passage and into a patient's airway. The pacifier may be resilient and may be softer than the oropharyngeal airway. The pacifier may be removably coupled to the artificial airway. The pacifier may encircle, to varying degrees, various portions of the artificial airway. The pacifier may be sized so that no air may pass between the artificial airway and the patient's airway tissue. The pacifier and artificial airway may have numerous channels such as, for example, a first channel for conducting air and a second channel for aspirating fluids. The pacifier and/or artificial airway may come in sets that include variously sized pacifiers and/or artificial airways. The pacifier may also include a non-resilient plug that prevents the patient from fully closing its mouth and occluding its airway.

This application claims priority to U.S. Provisional Patent Application No. 60/927,629 filed on May 4, 2007 entitled ARTIFICIAL AIRWAY.

### BACKGROUND

An artificial airway, such as an oropharyngeal airway (OPA), may be used to maintain an open airway in a patient. The airway may, for example, include the mouth, pharynx, and tracheae. An OPA may be used during short term medical procedures while the patient is sedated and unable to properly ventilate without medical intervention. An OPA may also be used while attempting to sedate a patient. In other words, an OPA may be used during the early stages of sedation as the patient moves from a conscious state to a semiconscious state or unconscious state. In some instances, the OPA may later be replaced with an endotracheal tube. The OPA may prevent the patient's tongue from covering the epiglottis and consequently obstructing the patient's airway and breathing.

Use of OPAs with infant humans may present several problems. Infants may have excess tissue (e.g., swollen tongue) that complicates insertion of the OPA into the child. In addition, the child's gag reflex may also complicate OPA insertion efforts. Furthermore, insertion of the OPA into the child may lead to spasms in the infant. As a result, clinicians often must wait to insert the OPA into the infant until after the infant has been thoroughly sedated.

### BRIEF DESCRIPTION OF THE DRAWINGS

The accompanying drawings, incorporated in and constituting a part of this specification, illustrate one or more implementations consistent with the principles of the invention and, together with the description of the invention, explain such implementations. The drawings are not necessarily to scale, the emphasis instead being placed upon illustrating the principles of the invention. In the drawings:

FIG. 1 includes a side view of a device in one embodiment of the invention.

FIG. 2 includes a front view of a device in one embodiment of the invention.

FIG. 3 includes a side view of a device in one embodiment of the invention.

FIG. 4 includes a side view of a device in one embodiment of the invention.

FIG. 5 includes a front view of a device in one embodiment of the invention.

### DETAILED DESCRIPTION

The following description refers to the accompanying drawings. Among the various drawings the same reference numbers may be used to identify the same or similar elements. While the following description provides a thorough understanding of the various aspects of the claimed invention by setting forth specific details such as particular structures, architectures, interfaces, and techniques, such details are provided for purposes of explanation and should not be viewed as limiting. Moreover, those of skill in the art will, in light of the present disclosure, appreciate that various aspects of the invention claimed may be practiced in other examples or implementations that depart from these specific details. At certain junctures in the following disclosure, descriptions of well known devices, circuits, and methods have been omitted to avoid clouding the description of the present invention with unnecessary detail.

One embodiment of the invention includes an oropharyngeal airway and a pacifying device. The pacifying device may sooth the infant, thus making it possible to introduce the oropharyngeal airway into the infant's airway before the infant is sedated or early/earlier in the sedation process.

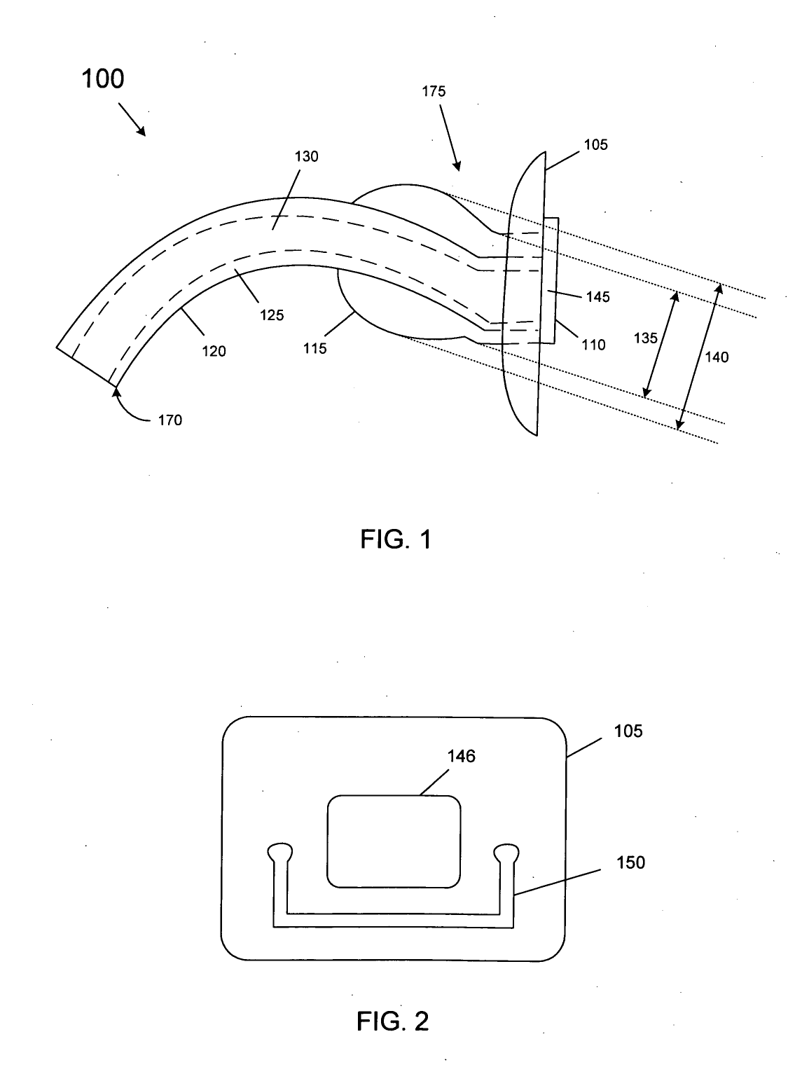

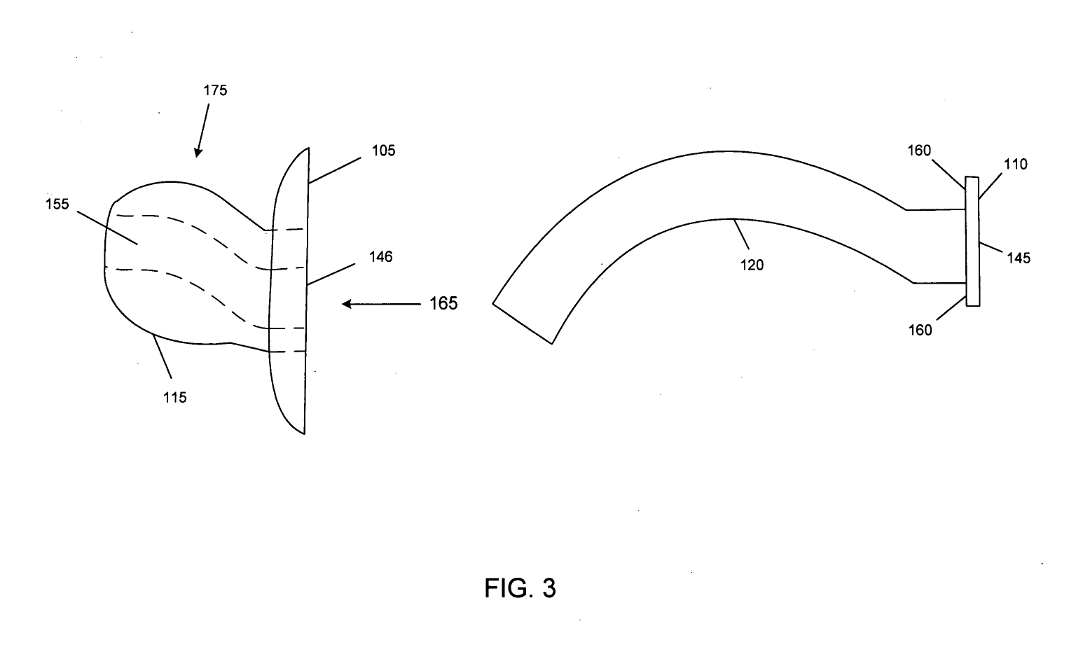

FIG. 1 includes a side view of a device 100 in one embodiment of the invention. The device includes an artificial oropharyngeal airway 120. The airway 120 includes a wall 125 and a passage 130. The passage 130 may be used for, as an example, ventilation or removal of fluids and the like from the patient. In one embodiment of the invention, the wall 125 has a first hardness. In other words, the wall 125 may be composed of a firm, stiff, hard or, in some embodiments of the invention, non-resilient material (e.g., polyethylene) that may ensure the airway passage 130 does not collapse under force applied by, for example, an infant's bite or a child's bite. The passage 130 terminates at an opening 145 formed in the surface 110 (FIGS. 1 and 3), located at the proximal end of the airway 120, and at an opening 146 (FIGS. 2 and 3) formed in the pacifier 175. The passage 130 also terminates at the distal portion 170 of the airway 120.

FIG. 3 includes a side view of one embodiment of a pacifier. Referring to FIGS. 1 and 3, the airway 120 may be received within a conduit 155 of a device such as an infant's pacifier 175. The pacifier 175 may include a surface 105 that rests against the child's face when the airway is inserted into the child. The surface 105 (e.g., face guard) may help prevent the child from swallowing the device 100 and may act to soothe the infant. The pacifier portion may include a bulb 115. The bulb may include a second hardness. The second hardness may be resilient (e.g., silicon) in imitation of a human nipple. The bulb 115 may contour or narrow as it approaches surface 105. In other words, the bulb 115 may include at least two diameters 135, 140, or alternatively a graduated diameter, with the diameter 140 near the surface 105 being smaller than the diameter 135 that is further removed from surface 105. Alternative embodiments of the invention may include analogous structures to the bulb. In other words, a resilient member may take a form other than a bulbous form.

In one embodiment of the invention, the pacifier portion 175, including the surface 105 and bulb 115, surrounds at least a portion of the oropharyngeal airway 120. In other embodiments, the bulb may extend to (not shown) or past (not shown) the distal portion 170 of the airway 120. The diameter of the bulb 115 near the distal portion 170 of the airway 120 may be narrowed so that room may exist between the bulb 115 and the infant's own airway walls. However, in other embodiments the diameter may be broadened to prevent any spacing between the bulb 115 and the child's airway walls. This lack of space may allow the practitioner more complete control over the infant's breathing.

In one embodiment of the invention, the airway 120 and pacifier 175 are uniform. For example, the entire device 100 may be permanently coupled together. For example, the entire device 100 may be formed from a single die. As another example, the airway 120 may be permanently coupled to the pacifier 175 using a permanent adhesive.

FIG. 2 includes a front view of a device in one embodiment of the invention. Opening 146 in surface 105 may be coupled (not shown) to the passage 130 of the airway 120 (FIG. 1). A handle 150 may be coupled to surface 105 to facilitate handling and general removal of the device 100 from the infant's airway.

FIG. 3 includes a side view of a device in one embodiment of the invention. The pacifier 175 is separable from the airway 120 in this particular embodiment of the invention. The pacifier 175 and airway 120 may be coupled to one another by inserting the airway 120 into the passage 155 of the bulb 115 in the general direction 165. This coupling may occur, for example, before or after the pacifier is given to the infant. Thus, in one embodiment of the invention, a standard airway (e.g., item 120) may be inserted into the pacifier 175 to provide an oral airway that is more tolerable to an infant. The airway 120 may be slid into the passage 155 until surface 160 abuts surface 105. The airway 120 may be further sufficiently held within passage 155 due to the friction that may reside between passage 155 and the airway 120. Of course, in some embodiments of the invention the passage 155 may include rails that cooperate with channels in the airway 120 to further couple the two elements together. In one embodiment of the invention, the bulb 115 may include a resilient material such as, for example, silicon or rubber. The airway may include a non-resilient plastic such as, for example, polyethylene.

Thus, in one embodiment of the invention, the airway 120 is inserted into the pacifier 175. The airway 120 and pacifier 175 are then jointly introduced to the infant or child. In another embodiment of the invention, the pacifier 175 is first introduced to the infant or child. The airway 120 is then introduced, via the passage 155 of the pacifier 175, to the infant or child.

In one embodiment of the invention, the airway 120 may include more than one passage. For example, one passage may exist for ventilation while another passage may be used to remove mucus. However, a single passage may be used for both ventilation and mucus removal or the like.

In one embodiment of the invention, the surface 105 may be of minimal size or may be nonexistent. In such a scenario, the bulb 115 may be coupled to the airway 120 using an adhesive or may be of uniform construction with the airway 120. The minimal or nonexistent surface 105 may provide the clinician with better access to the infant's airway and oral cavity.

In one embodiment of the invention, the bulb 115 and airway 120 may have the same hardness. For example, each may be resilient. In some embodiments of the invention, the bulb 115 and airway 120 may each be resilient but to varying degrees. For example, the bulb 115 may be softer than airway 120, although both bulb 115 and airway 120 are resilient to some degree.

In one embodiment of the invention, the devices 100 may be available as a set that includes a plurality of differently sized airways 120. The differently sized airways 120 may include sizes associated with traditional OPA's (e.g., #0, 50 mm). The pacifier unit 175, whether separable or uniform with the airway 120, may range in size or be available in a single size for use with the variably sized airways 120.

Thus, in one embodiment of the invention, the pacifier 175 may sooth the infant, thus making it possible to introduce the airway 120 into the infant's airway before the infant is sedated. The pacifier 175 may allow for introduction of the airway while the infant is awake and fully conscious. In other words, the airway 120 is inserted into the pacifier 175 and then into the patient. The patient may better tolerate the airway 120 due to soothing effect of the pacifier 175. However, in another embodiment of the invention, the pacifier 175, without airway 120, may be first introduced to the infant prior to introducing the airway 120 to the infant. Afterwards, after the patient is soothed by the pacifier 175, the airway 120 may be introduced to the infant through the passage 155 of the pacifier 175.

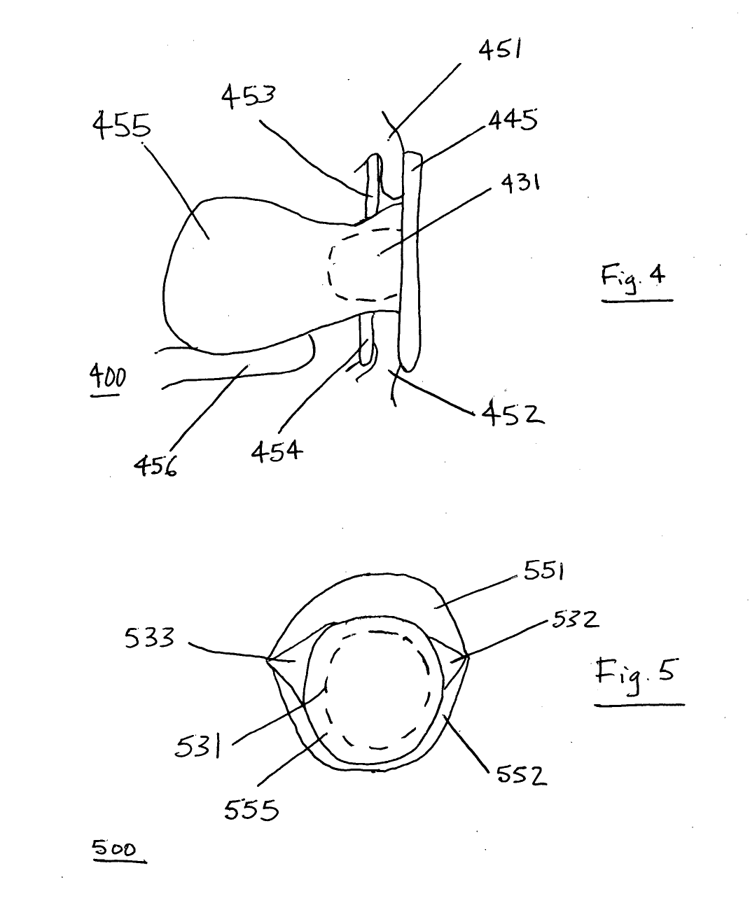

FIG. 4 includes a side view of a device 400 in one embodiment of the invention. A bulbous region 455 may include a plug 431. The bulbous region 455 may be resilient in a manner similar to the bulbous region of traditional pacifiers. Bulbous region 455 may be hollow in one embodiment of the invention. However, the invention is not limited in this manner. Plug 431 may he hard and non-resilient. In other embodiments, only a portion of the plug 431 may be nonresilient or substantially nonresilient. Plug 431 may be solid but may also be hollow or include a void. Thus, if a child bites down with an upper tooth 453 and bites up with a bottom tooth 454, the teeth 453, 454 and lips 451, 452 will not be able to meet. The teeth 453, 454 may directly contact plug 431 or may indirectly couple to plug 431 via a softer material connected to bulbous portion 455. As shown in FIG. 5, the failure for the teeth 453, 454 and lips 451, 452, 551, 552 to respectively meet may result in maintaining an open airway for the child. In other words, due to the hard plug 431, 531, a child cannot fully close its mouth. This may lead to spaces 532, 533 existing on either side of the device. A health care practitioner can then easily insert a suction tube down the side of the mouth using either or both spaces 532, 533. Furthermore, the child or patient may be able to breathe using spaces 532, 533. The device may include a surface 445, 555 that extends and/or resides outside, for example only, the mouth and lips 451, 452, 551, 552. This prevents the child from swallowing the device or having the device recede too far into the child's airway. Surface 445, 555 may have varying forms. Surface 445, 555 may be smaller than face guards found on traditional pacifiers. The smaller profile of surface 445, 555 may better allow a practitioner access to, for example, spaces 532, 533. The surface may be tapered or pinched inward on its sides. The pinched portion of the profile may enlarge spaces analogous to spaces 532, 533. The top and bottom portions may be elongated to further prevent the device from sliding too far into the airway.

In various embodiments of the invention, the device may not include a plug but instead may merely include a portion that is harder than other portions of the device. That hard portion may be formed so that it naturally fits between the child's teeth thereby ensuring the child does not occlude its own airway by clinching his or her teeth and mouth shut. The hard region may extend the entire length of device 400 (e.g., from the distal tip of bulbous region 455 to the proximal tip of face plate 445) or may extend only a portion of the entire length of the device 400. In one embodiment of the invention, bulbous portion 455 may extend no further into the airway than does a typical infant pacifier. In this light, the device 400 may be easily tolerated by an unsedated or partially sedated patient. In other words, bulbous region 455 extends into the airway without extending into the trachea or vocal cords in a manner similar to traditional pacifiers (which are tolerated by infants). However, bulbous region 455 may extend far enough into the airway to prevent the tongue from occluding the airway. For example, tongue 456 may rest under bulbous region 455. Of course, bulbous region 455 need not have a bulbous profile and may incorporate varying geometries without limitation (e.g., spoon, disk, planar, oblong, etc.).

Thus, in one embodiment of the invention a pacifier includes an opening (e.g., void, recess, conduit, passage, channel, hole). An artificial airway (e.g., oropharyngeal airway) may be inserted in a patient's airway via the opening. The artificial airway may extend to varying degrees within the airway (e.g., (1) to the mouth, (2) to the mouth and vocal cords, (3) to the mouth, vocal cords and trachea). The opening may have a hardness that is softer than a hardness of the artificial airway. In other embodiments, the hardness of the opening may be the same as or harder than the hardness of the artificial airway. The pacifier and opening may be separable from the artificial airway. However, the pacifier and opening may be integral with and permanently coupled to the artificial airway. The opening may encircle (like a ring) varying degrees or portions of the artificial airway. For example, the artificial airway may have a distal portion that is inserted deepest into the patient airway. The pacifier opening may extend distally all the way to the distal most end or tip of the artificial airway. Of course in other embodiments the pacifier opening may not extend so far. In various embodiments of the invention the artificial airway may extend to varying degrees within the airway. The artificial airway may extend into the mouth. In other embodiments the artificial airway may extend into the mouth and beyond (i.e., into/beyond the pharynx, the trachea, etc.). A channel (e.g., for fluid aspiration) may be coterminous with and substantially parallel to the artificial airway. This channel may be included in the pacifier. However, it may also be included in or coupled to the artificial airway itself. The pacifier may include a bulb, where the bulb includes the opening. The bulb may taper near the proximal end (i.e., end near the mouth) of the artificial airway. The bulb may extend various distances down (i.e., distally) the artificial airway. The bulb may have varying breadths. A portion of the bulb may have a breadth (i.e., diameter) sized such that the bulb seals the space between the artificial airway and the patient's airway.

In an embodiment of the invention a user may introduce a pediatric pacifier, having a first opening, to an infant and insert an artificial airway into the infant's airway via the first opening. The pacifier may already be introduced to the patient at the time the artificial airway is introduced to the pacifier opening. Of course, the artificial airway may be coupled to the pacifier before either the pacifier or the artificial airway is introduced to the patient. In some embodiments, the pacifier may be slipped over an artificial airway that is already introduced to the patient. The patient (e.g., an infant) may be partially sedated, fully sedated, or unsedated upon introduction of artificial airway and/or pacifier. The artificial airway may be removed from the first opening and replaced with an endotracheal tube. This exchange may occur with or without removing the pacifier from the infant.

In an embodiment of the invention, a device may include a pacifier alone. The pacifier may include a first opening (e.g., channel, void, conduit, space) that can receive an “of the shelf” or “standard” artificial airway. The infant pacifier may include a portion (e.g., bulb) that will be coterminous (end at the same point) with an artificial airway after that artificial airway is fully inserted in or coupled to the pacifier. The pacifier may be resilient whereas the artificial airway may be non-resilient.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. An apparatus comprising:
a pacifier, having a first hardness, including a conduit; and
an oropharyngeal airway, having a second hardness, including a proximal portion, and a distal portion;
wherein the first hardness is softer than the second hardness and at least a first portion of the distal portion of the oropharyngeal airway is to insert, via the pacifier conduit, into a patient's airway.

2. The apparatus of claim 1, wherein the pacifier is to removably couple to the oropharyngeal airway.

3. The apparatus of claim 1, wherein the conduit is to encircle at least a first portion of the proximal portion of the oropharyngeal airway.

4. The apparatus of claim 3, wherein the conduit is to encircle at least the first portion of the distal portion of the oropharyngeal airway.

5. The apparatus of claim 3, further comprising an additional conduit to couple the pacifier to the patient's airway.

6. The apparatus of claim 1, wherein the pacifier includes a bulbous portion, the bulbous portion includes the conduit, and the bulbous portion tapers near a proximal end of the oropharyngeal airway.

7. A method comprising:
inserting a pediatric pacifier, having a first opening, into an infant's airway; and
inserting an artificial airway into the infant's airway via the first opening of the pediatric pacifier.

8. The method of claim 7, further comprising inserting the pediatric pacifier into the infant's airway before inserting the artificial airway into the infant's airway via the first opening of the pediatric pacifier.

9. The method of claim 7, further comprising inserting the pediatric pacifier into the infant's airway while inserting the artificial airway into the infant's airway via the first opening of the pediatric pacifier.

10. The method of claim 7, further comprising inserting the pediatric pacifier into the infant's airway and inserting the artificial airway into the infant's airway via the first opening of the pediatric pacifier before fully sedating the infant.

11. An apparatus comprising an infant pacifier that includes a first opening; wherein an artificial airway is to insert into a patient's airway via the first opening-of the infant pacifier.

12. The apparatus of claim 11, wherein the infant pacifier includes a first portion to insert into the patient's airway, the first portion including the first opening and having a first hardness that is softer than the external surface of the artificial airway.

13. The apparatus of claim 11, further comprising the artificial airway.

14. The apparatus of claim 11, wherein the infant pacifier includes a first portion to insert into the patient's airway, the first portion being resilient.

15. An apparatus comprising:
a pacifier, including a first portion having a first hardness and a second portion having a second hardness; wherein the first hardness is softer than the second hardness, at least a third portion, included in the first portion, is to insert into a patient's airway, and the second portion is to insert into the patient's airway and separate the medial portion of the patient's upper lip from the medial portion of the patient's lower lip.

16. The apparatus of claim 15, wherein the third portion is to be located distally to the second portion.

17. The apparatus of claim 16, wherein the first hardness is resilient and the second hardness is non-resilient.

18. The apparatus of claim 16, wherein the first portion is to couple to a top portion of the patient's tongue.

19. The apparatus of claim 16, wherein the second portion is to separably couple to the first portion.

20. The apparatus of claim 16, wherein the pacifier is coupled to a first surface, the first surface to be located outside the patient's airway and to connect to the patient's lips.
